(19) United States
(12) Patent Application Publication
(10) Pub. No.: US 2026/0199105 A1
(43) Pub. Date: Jul. 16, 2026

(54) ADAPTIVE DUAL-PATH NEURAL SIGNAL PROCESSING METHOD AND COMPUTING DEVICE FOR BRAIN-COMPUTER INTERFACE SYSTEMS

(71) Applicant: Homatch.ai, Milpitas, CA (US)

(72) Inventor: Mingjun Wang, Saratoga, CA (US)

(21) Appl. No.: 19/448,022

(22) Filed: Jan. 13, 2026

Related U.S. Application Data
(60) Provisional application No. 63/745,225, filed on Jan. 14, 2025.
(60) Provisional application No. 63/745,765, filed on Jan. 15, 2025.
(60) Provisional application No. 63/843,149, filed on Jul. 13, 2025.

(51) Int. Cl. A61F 2/72; G06F 3/01; G06N 3/004

(52) U.S. Cl. CPC A61F 2/72; G06N 3/004; G06F 3/015

(57) ABSTRACT

A method performed by a computing device for processing neural signals associated with biological brain activities and a corresponding computing device are provided. The method includes separating neural signals into components associated with different temporal characteristics and processing such components in parallel using different processing paths. By applying different processing paths that are configured to operate on neural signal components associated with distinct temporal dynamics, the disclosed method improves prediction precision, enhances feature extraction performance, and optimizes overall decoding accuracy in brain-computer interface systems.

### CROSS-REFERENCE TO RELATED APPLICATIONS

This application claims priority to U.S. Provisional Patent Application Ser. No. 63/745,225, filed Jan. 14, 2025, entitled “A TWO PATH, BIDIRECTIONALLY COUPLED NEURAL SIGNAL PROCESSING METHOD AND APPARATUS”; U.S. Provisional Patent Application Ser. No. 63/745,765, filed Jan. 15, 2025, entitled “BRAIN-COMPUTER INTERFACE CHIP BASED ON A DOUBLE-HELIX AND ITS SIGNAL PROCESSING METHOD” and U.S. Provisional Patent Application Ser. No. 63/843,149, filed Jul. 13, 2025, entitled “DUAL PATH CNN LSTM ADAPTIVE BRAIN COMPUTER INTERFACE CHIP AND REAL TIME SIGNAL PROCESSING METHOD”. The contents of these applications are hereby incorporated by reference in their entirety for all purposes.

### TECHNICAL FIELD

This disclosure relates to brain-computer interface (BCI) technology, including techniques for processing neural signals with different processing paths.

### BACKGROUND

Brain-computer interface (BCI) technology enables direct communication between neural activity and external computing or control systems. BCIs typically operate by acquiring, interpreting, and translating neural signals generated by the brain into commands that may be used to control external devices, perform assistive functions, or provide feedback to the user. Neural signals may be recorded using a variety of sensing modalities, including non-invasive techniques such as electroencephalography (EEG), magnetoencephalography (MEG), or functional near-infrared spectroscopy (fNIRS), as well as invasive or semi-invasive techniques such as electrocorticography (ECOG) or microelectrode arrays capable of detecting neuronal spikes.

A challenge for brain-computer interface (BCI) systems arises from the inherently multi-dimensional and temporally dynamic nature of neural data. Neural signals may vary across multiple dimensions, including frequency content, amplitude, energy distribution, and spatiotemporal structure, and such characteristics may change over time, across cognitive or physiological states, and between different individuals. In certain examples, neural signals may include components spanning a broad range of temporal or spectral scales, with different components conveying distinct informational content related to cognitive activity, motor intention, or other neural processes.

### BRIEF DESCRIPTION OF THE DRAWINGS

FIG. 1 is a flowchart illustrating an example method for processing neural signals associated with biological brain activities in accordance with embodiments of the present disclosure.

FIG. 2 is a flowchart illustrating another example method for processing neural signals associated with biological brain activities in accordance with embodiments of the present disclosure.

FIG. 3 is a block diagram illustrating an example computing device for processing neural signals associated with biological brain activities in accordance with embodiments of the present disclosure.

### DETAILED DESCRIPTION

Aspects of the present disclosure are directed to a method performed by a computing device for processing neural signals associated with biological brain activities and to a corresponding computing device. The method includes separating neural signals into components associated with different temporal characteristics and processing such components in parallel using different processing paths (e.g., a first processing path and a second processing path). By applying different processing paths that are configured to operate on neural signal components associated with distinct temporal dynamics, the disclosed method can improve prediction precision, enhance feature extraction performance, and optimize overall decoding accuracy.

Although convolutional neural networks, recurrent neural networks, long short-term memory networks, and similar machine-learning architectures are described in certain embodiments, the dual-path signal processing and bidirectional feedback mechanisms disclosed herein are not limited to any particular computational model, neural network architecture, or learning paradigm. Other statistical, signal-processing, or machine-learning techniques may be used to implement the disclosed functionality without departing from the scope of the invention.

Brain-computer interface (BCI) technology generally refers to systems configured to enable communication between a user's neural activity and an external device. The external device may include, but is not limited to, a robotic or prosthetic limb, a wheelchair or mobility-assist robot, a computer cursor or typing interface, a virtual-reality or augmented-reality control system, or a neurostimulator used for therapeutic modulation. A BCI system typically acquires neural signals from the brain through invasive or non-invasive sensing modalities, such as electroencephalography (EEG), electrocorticography (ECOG), intracortical microelectrodes, or other neural recording techniques. The acquired neural signals may be processed to extract features associated with cognitive states, motor intentions, sensory responses, or other brain activities, and the extracted features may be translated into control commands for operating external devices or software applications. BCI technology has been applied in various fields, including assistive robotics, neuroprosthetics, medical rehabilitation, communication aids, and human-machine interaction. However, the multi-dimensional, non-stationary, and highly individualized nature of neural signals presents challenges for reliable decoding and accurate prediction of user intent.

BCI systems may employ artificial intelligence (AI) models to process neural signals in a unified or sequential manner without explicitly distinguishing between neural signal components associated with different temporal characteristics. Such single-model systems may lack processing paths optimized for both rapidly varying neural activity and more slowly evolving neural dynamics, leading to reduced decoding performance in dynamic neural environments. As a result, such systems may exhibit increased latency or reduced decoding fidelity under varying conditions, such as noise, non-stationary signal behavior, or changes in user state.

To address these and other issues, aspects of the present disclosure provide a computing device and a method performed by the computing device. The present disclosure introduces a dual-path processing method and device that separates neural signals into components associated with relatively short-term neural dynamics and components associated with relatively long-term neural dynamics, and processes such components in parallel using different processing paths. The first processing path is configured to generate at least one first feature indicative of short-term neural activity, and the second processing path is configured to generate at least one second feature indicative of long-term neural activity. By applying different processing paths that are tailored to different temporal characteristics of the neural signals, the disclosed method can improve prediction precision, enhance feature extraction performance, and optimize overall decoding accuracy. Such separation and parallel processing allow each processing path to focus on different aspects of neural signal behavior and reduce interference between disparate neural dynamics.

Additionally, the present disclosure incorporates dynamic feedback for parameter adjustment. Specifically, the computing device may compare the at least one first feature generated by the first processing path and the at least one second feature generated by the second processing path to generate a feedback signal and adjust at least one parameter associated with the first processing path and the second processing path based on the feedback signal. This dynamic feedback may be bidirectional, such that refined feature representations generated by one processing path may be provided to the other processing path to influence subsequent processing. Such bidirectional feedback enables mutual refinement between the two processing paths and enhances cross-domain feature alignment during operation, thereby coupling short-term and long-term signal representations in a manner that adapts system behavior in real time and improves overall performance of the computing device.

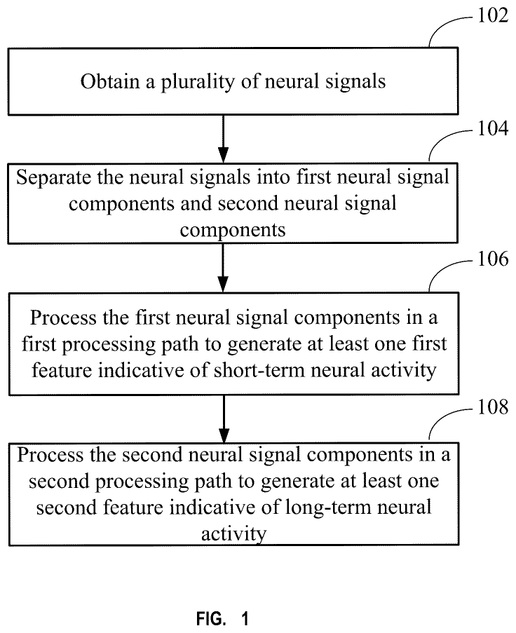

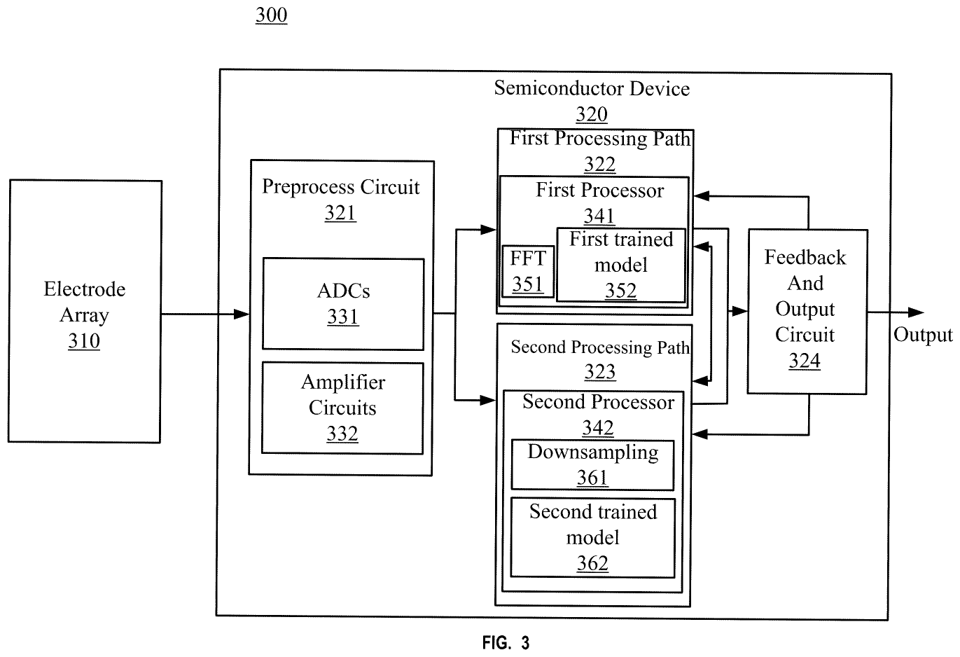

According to an aspect of the present disclosure, this disclosure first discloses a method performed by a computing device for processing neural signals associated with biological brain activities. FIG. 1 illustrates flowcharts showing a method 100 for processing neural signals associated with biological brain activities in accordance with examples disclosed herein. The method 100 implements a dual-path processing framework that separates neural signals into components associated with different temporal characteristics, processes them independently, and integrates their outputs to determine a neural activity pattern representative of both short-term and long-term neural activities. The method 100 can be performed by a computing device (e.g., computing device 300 described below with reference to FIG. 3). As shown and will be described in FIG. 3, the computing device includes a first processing path configured to process neural signal components associated with relatively short-term dynamics and a second processing path configured to process neural signal components associated with relatively long-term or slowly varying dynamics.

At block 102 of FIG. 1, the computing device obtains a plurality of neural signals from one or more neural signal acquisition devices. The computing device may receive raw neural data corresponding to electrical or electrophysiological activity measured from a subject. In various embodiments, the neural signals may originate from electroencephalography (EEG), electrocorticography (ECOG), local field potentials (LFPs), intracortical recordings, or other neural sensing modalities.

The one or more acquisition devices may include multi-channel electrode arrays, which may be non-invasive, minimally invasive, or invasive. In certain non-limiting examples, the acquisition devices may comprise an electrode array including a plurality of electrodes disposed at different locations relative to a subject's neural tissue to obtain a plurality of neural signals. The electrodes may be positioned on or near a scalp surface, cortical surface, or within neural tissue, depending on the sensing modality employed.

Each electrode or channel may capture voltage or current fluctuations associated with neural activity, including neuronal firing or aggregate neural dynamics. The neural signals may be sampled at one or more sampling rates selected according to the sensing modality and application, which may range from relatively low sampling rates suitable for surface recordings to higher sampling rates suitable for capturing rapid neural events. The specific number of channels, electrode configuration, and sampling rates are implementation-dependent and are not intended to limit the scope of the invention.

In one non-limiting example, the acquisition devices may include an electroencephalography (EEG) cap comprising a multi-channel electrode array. The EEG cap may be used to record neural activity during a motor imagery task, in which a subject mentally rehearses a movement without producing corresponding physical motion. The recorded neural activity may be represented as a set of time-varying signals x_i(t), where i denotes a channel index and t denotes time.

The computing device may obtain a multi-dimensional digital representation of the neural signals over a selected time interval and sampling rate, resulting in a data structure having dimensions corresponding to a number of channels and a number of time samples. The specific number of channels, sampling frequency, and duration of the recording interval are implementation-dependent and may be selected based on the sensing modality, task type, or application requirements. Such representations are provided for illustrative purposes and are not intended to limit the scope of the invention.

At block 104, the computing device separates neural signal components corresponding to different temporal or spectral characteristics. The obtained neural signals may contain signal components associated with relatively rapid variations as well as components associated with slower or more sustained variations. In various embodiments, the computing device separates the neural signals into first neural signal components representing short-term or rapidly varying neural activity and second neural signal components representing longer-term or more slowly varying neural activity.

The first neural signal components may include signal features indicative of transient or localized neural events, such as neural spiking activity, high-frequency oscillatory activity, or other rapidly changing signal patterns. The second neural signal components may include signal features indicative of broader or sustained neural dynamics, such as slow cortical potentials, rhythmic or oscillatory activity, or trends reflecting evolving cognitive or physiological states.

In one non-limiting illustrative example only, the first neural signal components may correspond to relatively higher-frequency signal content and the second neural signal components may correspond to relatively lower-frequency signal content. For example, higher-frequency signal content may be associated with transient or localized neural activity, while lower-frequency signal content may be associated with slower or more sustained neural dynamics. The specific frequency ranges described herein are illustrative and implementation-dependent and are not intended to limit the scope of the invention.

The separation of neural signal components may be performed using frequency-domain, time-domain, time-frequency, or learned signal decomposition techniques. The specific frequency ranges or temporal scales associated with the separated components are implementation-dependent and may vary according to sensing modality, application, or subject characteristics. The separation is not limited to fixed frequency cutoffs and instead reflects a distinction between neural signal components associated with different temporal abstractions.

In various embodiments, the separation of neural signal components associated with different temporal or spectral characteristics may be achieved using one or more signal decomposition or filtering techniques. For example, the computing device may apply digital filtering, adaptive filtering, or multi-resolution analysis to extract signal components corresponding to relatively rapid variations and signal components corresponding to slower or more sustained variations.

In non-limiting examples, filtering operations may include high-pass, low-pass, band-pass, or band-stop filters having cutoffs selected according to the sensing modality, application context, or observed signal characteristics. In other embodiments, time-frequency or multi-scale decomposition techniques may be employed, including wavelet-based methods, short-time Fourier transform (STFT), filter banks, empirical mode decomposition, or learned spectral decomposition models.

The specific filtering parameters, decomposition techniques, and cutoff values are implementation-dependent and may be dynamically adjusted or learned during operation.

Accordingly, the separation of neural signal components is not limited to fixed frequency boundaries and instead reflects a distinction between neural signal components associated with different temporal abstractions.

At block 106, the computing device processes the first neural signal components associated with relatively short-term dynamics in a first processing path (also referred to as a local processing path) to generate at least one first feature. The first processing path is dedicated to analyzing the short-term neural signal components to extract features indicative of transient or localized neural activity.

In a first processing path (also referred to as a local path), the neural signal components associated with relatively rapid or short-term variations may be provided as input to a feature extraction component, which may comprise a convolutional neural network (CNN) or another trained or untrained feature extraction model. The feature extraction component is configured to identify temporal, spatial, or spatiotemporal patterns across channels that are indicative of transient or localized neural activity, such as rapid modulations, bursts, or other short-duration signal features.

In certain non-limiting examples, a convolutional neural network may apply convolutional operations across time and channel dimensions to detect localized micro-patterns within the short-term signal components, such as bursts or synchronized oscillatory activity. The feature extraction component generates one or more first feature representations based on the detected patterns. These feature representations may encode information such as event density, burst characteristics, localized activation levels, or other representations of short-term neural dynamics.

The specific structure, depth, and parameters of the feature extraction component are implementation-dependent and are not intended to limit the scope of the invention. Other feature extraction mechanisms capable of capturing short-term or localized signal characteristics may be used in place of or in combination with convolutional neural networks.

At block 108, the computing device processes the second neural signal components associated with relatively long-term dynamics in a second processing path (also referred to as a global processing path) to generate at least one second feature. The second processing path is configured to derive features representing sustained neural activity or global brain states.

In a second processing path (also referred to as a global path), the neural signal components associated with longer-term or more slowly varying dynamics may be provided to a temporal modeling component configured to capture extended temporal dependencies in the neural signals. The temporal modeling component may comprise a recurrent neural network, such as a Long Short-Term Memory (LSTM) model, or another temporal inference mechanism capable of modeling sequential patterns over extended durations.

The temporal modeling component processes sequences of neural signal representations over one or more input windows selected to reflect longer-term neural dynamics, such as sustained cognitive or physiological states. The temporal modeling component generates one or more second feature representations that characterize global or slowly evolving neural activity, which may correspond to overall brain states, contextual conditions, or longer-term intentions.

The specific structure, duration of input windows, internal state representations, and parameterization of the temporal modeling component are implementation-dependent and may vary according to sensing modality, application requirements, or subject characteristics. Accordingly, the global processing path is not limited to any particular recurrent architecture, gating mechanism, or temporal window size.

It should be noted that, although in the illustrated example the processing of neural signal components associated with short-term dynamics (e.g., block 106) is shown as occurring prior to the processing of neural signal components associated with long-term dynamics (e.g., block 108), no limitation is intended regarding the order of these operations. In other embodiments, the long-term components may be processed before the short-term components, or the two processing paths may be executed concurrently, iteratively, or in parallel, depending on system design or implementation preferences.

After the first and second features are generated, a neural activity pattern may be determined based on the at least one first feature and the at least one second feature. The computing device may determine the neural activity pattern based on the outputs of both processing paths. For example, the computing device may fuse the outputs of the two processing paths to generate a fused feature representation or feature matrix. The computing device may then use a classification or inference model to determine the neural activity pattern based on the fused feature representation. Alternatively, the computing device may transmit the outputs of one or both processing paths to an external device for further processing. The neural activity pattern may be further used in various implementations, including, but not limited to, motor control, cognitive monitoring, and medical diagnosis.

Motor control refers to brain activity related to planning and performing physical movements, such as moving a left or right limb. Neural signals associated with motor control may include both transient, localized patterns (such as spike-like events or rapidly changing activity) and slower rhythm changes that occur before or during movement. In a motor control implementation, the computing device may communicate with an external device, such as an exoskeleton or assistive robotic system, which may be mounted on or coupled to a user and configured to assist movement based on the user's neural activity. For example, neural signals corresponding to an intended movement may be processed as described above to generate feature representations or fused outputs, which may be transmitted to the external device to determine a neural activity pattern and control the external device accordingly.

Cognitive monitoring refers to detecting mental or physiological states such as alertness, drowsiness, or fatigue. Such states are often reflected by slower and more global neural activity changes that evolve over longer time periods and may be captured by the processing of neural signal components associated with longer-term dynamics.

As described above, a classification or inference model may be used to determine the neural activity pattern. This determination process may involve fusing the first and second feature representations and using a classification head or decision module to map the fused features to a defined brain state, intent category, or control command. In some implementations, the fused feature representations may be further processed, stored, or transmitted for subsequent analysis or decision-making. The fusion and classification operations are described in greater detail below.

The neural activity pattern may also be applied to medical diagnostic systems. Such systems may analyze the neural activity pattern for early detection, monitoring, or assessment of neurological conditions. For example, the first processing path may extract transient neural patterns associated with abnormal events, while the second processing path may capture slower-varying neural dynamics indicative of broader neurological conditions. A fused feature representation generated from both processing paths may enable a diagnostic engine to identify pathological neural signatures with improved sensitivity and specificity.

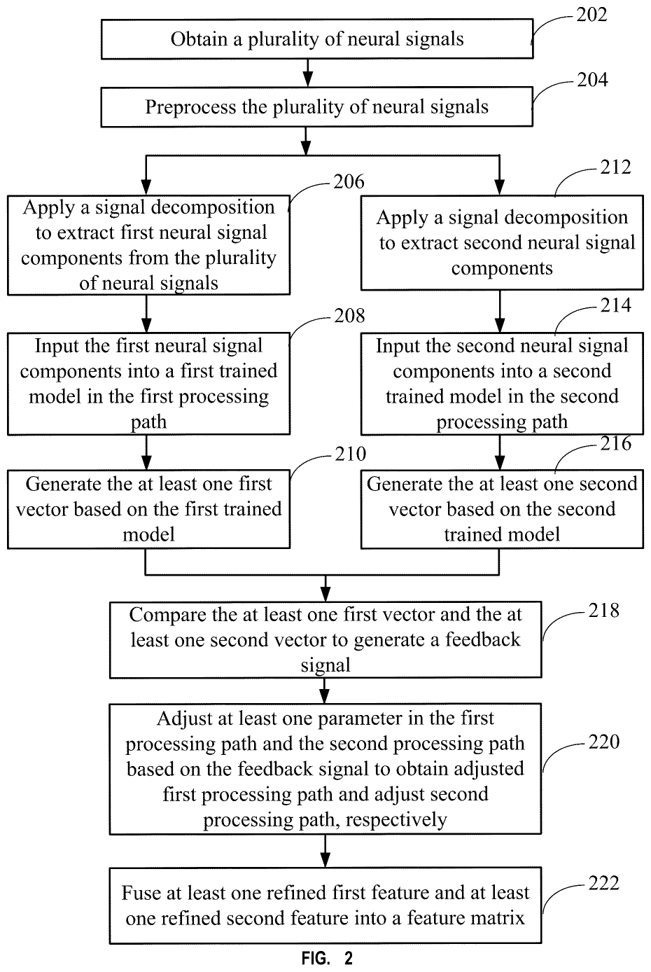

FIG. 2 illustrates flowcharts showing another example method 200 for processing neural signals in accordance with embodiments disclosed herein. The method 200 may be performed by a computing device, such as the computing device 300 described below with reference to FIG. 3. As shown in FIG. 2, block 202 corresponds to an operation similar to that shown at block 102 in FIG. 1, and a detailed description of that operation is therefore omitted for brevity.

In some embodiments, as shown at block 204 of FIG. 2, the computing device may preprocess the plurality of neural signals to mitigate noise or interference prior to further processing. The preprocessing may include applying one or more signal conditioning operations configured to suppress noise components, artifacts, or undesired signal variations.

In various non-limiting examples, the computing device may apply digital filtering operations, such as band-pass, low-pass, high-pass, or notch filtering, to attenuate noise or interference associated with environmental sources, instrumentation artifacts, or physiological activity unrelated to the target neural signals. In other examples, the preprocessing may include adaptive or data-driven filtering techniques that dynamically adjust filtering parameters based on observed noise characteristics or signal quality metrics.

The preprocessing may further include spatial or multi-channel signal conditioning techniques, such as common-mode noise reduction, channel normalization, or other spatial filtering operations, to reduce correlated noise across multiple channels. The specific preprocessing techniques and parameters employed are implementation-dependent and may be selected or adjusted dynamically based on signal characteristics, sensing modality, or application requirements.

In some embodiments, referring to block 206 of FIG. 2, separating neural signal components associated with different temporal or spectral characteristics may include transforming the plurality of neural signals from a time-domain representation into an alternative representational domain suitable for distinguishing between relatively rapid and relatively slow signal variations. Such transformation may be performed using one or more spectral, time-frequency, or multi-scale analysis techniques.

In certain non-limiting examples, the computing device may apply a Fourier-based transform, such as a fast Fourier transform (FFT), to obtain a frequency-domain representation of the neural signals. The transformation may decompose the neural signals into a plurality of components corresponding to different spectral or temporal characteristics, which may be represented as frequency bins or equivalent representations. Other transform sizes, windowing strategies, or decomposition parameters may be used depending on application requirements, and the use of FFT is illustrative rather than limiting.

The use of a Fourier-based transform is illustrative only, and alternative transformation or decomposition techniques may be employed, including wavelet transforms, short-time Fourier transform (STFT), filter banks, empirical mode decomposition, or learned spectral representations. Accordingly, the separation of neural signal components is not limited to a specific transform type, transform size, or numerical configuration.

Once transformed or otherwise decomposed, the computing device may apply a signal selection or conditioning operation to isolate neural signal components associated with relatively rapid or transient neural events. Such operations may include thresholding, filtering, weighting, or selection of representations associated with rapid variations, while attenuating representations associated with slower or sustained dynamics. The resulting short-term neural signal representations may then be provided to the first processing path to generate at least one first feature representation.

In some embodiments, as described above, the first processing path, also referred to as a local path, is configured to process neural signal components associated with relatively rapid or short-term variations (e.g., the first neural signal components). The local path may be implemented using a trained feature extraction model, which may comprise a convolutional neural network (CNN) or another model capable of identifying localized temporal, spatial, or spatiotemporal patterns in the neural signals.

The computing device may provide representations of the short-term neural signal components to the trained feature extraction model in the local path. The trained feature extraction model generates one or more first feature representations, which may include one or more first vectors encoding characteristics of transient or localized neural activity.

In various embodiments, the input to the feature extraction model may be derived from segmented portions or windows of the short-term neural signal components, with window durations, dimensionality, and sampling characteristics selected according to application requirements, sensing modality, or signal characteristics. The segmentation strategy, window size, and input representation are implementation-dependent and are not limited to any particular duration, dimensionality, or frequency range.

Accordingly, the local path is not limited to a specific neural network architecture, window configuration, or numerical parameter set, and other feature extraction mechanisms capable of capturing short-term neural dynamics may be employed.

In some embodiments, the trained feature extraction model in the local processing path may comprise a neural network implemented using reduced-precision arithmetic, fixed-point arithmetic, or other computational formats suitable for efficient execution on resource-constrained computing devices. The feature extraction model may include one or more convolutional layers or equivalent processing stages arranged to extract localized temporal, spatial, or spatiotemporal features from the input neural signal representations.

In certain non-limiting examples, a convolutional layer may include a plurality of filter kernels configured to operate on input feature maps to extract local patterns across time, channels, or both. The convolutional layer may be followed by one or more nonlinear activation stages, normalization stages, pooling stages, or combinations thereof, to introduce nonlinearity, sparsity, or invariance into the extracted feature representations.

For example, a nonlinear activation function may suppress or attenuate selected signal components while preserving salient activations, thereby improving representational capacity and computational efficiency. Such activation functions may include rectified linear units (ReLU), leaky ReLU, sigmoid functions, hyperbolic tangent functions, or other nonlinear transformations. The specific network depth, numerical precision, activation functions, and parameterization are implementation-dependent and are not intended to limit the scope of the invention.

In some embodiments, intermediate feature representations generated by one or more feature extraction stages in the local processing path may be provided to additional processing stages configured to extract higher-level or more abstracted representations of the neural signals. Such additional processing stages may include one or more convolutional layers, aggregation layers, or equivalent transformation mechanisms arranged to progressively refine or abstract localized signal features.

The feature extraction stages may employ one or more nonlinear activation functions to introduce nonlinearity into the feature representations and enable modeling of complex relationships within the transformed neural signals. Nonlinear activation functions may include rectified linear units (ReLU), leaky ReLU, sigmoid functions, hyperbolic tangent functions, or other nonlinear transformations. The number of processing stages, the dimensionality of intermediate feature maps, and the specific activation functions employed are implementation-dependent and are not intended to limit the scope of the invention.

In some implementations, the feature extraction operations may be executed using hardware acceleration structures configured to efficiently perform repeated arithmetic operations, such as multiply-accumulate operations. Such hardware acceleration structures may include parallel processing arrays, vector processors, systolic arrays, or other specialized computing architectures suitable for executing feature extraction models. The use of hardware acceleration may improve processing efficiency, reduce latency, or increase throughput, but is not required for practicing the invention.

The resulting feature representations generated by the local processing path may be aggregated, flattened, pooled, normalized, or otherwise transformed to form one or more first feature vectors for use in subsequent processing stages. In certain embodiments, the feature extraction model may be trained using supervised, semi-supervised, or unsupervised learning techniques on one or more training datasets. The specific training datasets, optimization algorithms, parameter values, and training frameworks employed are implementation-dependent and are not intended to limit the scope of the invention.

In some embodiments, the feature extraction model may output a probabilistic or confidence-based representation associated with one or more neural activity categories or signal states. Such representations may include probability distributions, confidence scores, embeddings, or other numerical descriptors characterizing the short-term or localized neural activity. The at least one first feature vector may correspond to, be derived from, or incorporate such probabilistic or confidence-based representations.

In some embodiments, as described above, the second processing path, also referred to as a global path, is configured to process neural signal components associated with longer-term or more slowly varying dynamics (e.g., the second neural signal components). The global path may be implemented using a trained temporal modeling component capable of capturing extended temporal dependencies in neural signals, which may include a long short-term memory (LSTM) model or another sequential or temporal inference mechanism.

Referring to FIG. 2, at block 214, the computing device may provide representations of the longer-term neural signal components to the trained temporal modeling component in the global path. The longer-term neural signal components may be derived from temporally aggregated, down-sampled, or otherwise transformed representations of the original neural signals, using window durations and sampling characteristics selected to reflect sustained neural dynamics.

The temporal modeling component processes sequences of such representations over one or more extended input windows and generates one or more second feature representations that characterize global or slowly evolving neural activity. These second feature representations may correspond to broader neural states, contextual conditions, or longer-term cognitive or physiological patterns that may not be captured by the local processing path.

The specific temporal window sizes, sampling rates, dimensionality of input representations, frequency content, and internal structure of the temporal modeling component are implementation-dependent and may vary according to sensing modality, application requirements, or subject characteristics. Accordingly, the global processing path is not limited to any particular temporal resolution, sampling configuration, neural network architecture, or frequency range.

In some embodiments, the temporal modeling component in the global processing path may be implemented using a recurrent or sequential modeling architecture configured to process an input sequence over multiple time steps. Such a temporal modeling component may comprise one or more recurrent layers, state-based processing elements, or equivalent mechanisms that iteratively process sequential neural signal representations to capture long-range temporal dependencies.

In certain non-limiting examples, the temporal modeling component may include a long short-term memory (LSTM) model, in which a recurrent processing unit is applied repeatedly across a sequence of input representations. Each recurrent processing unit may maintain an internal state that is updated over time based on incoming signal representations and prior state information, thereby enabling the modeling of temporal context and longer-term dependencies within the neural signals.

In various embodiments, the recurrent processing units may include gating, attention, state-update, or memory mechanisms configured to regulate the incorporation of new information, the retention or attenuation of prior information, and the generation of output representations at each time step. Such mechanisms may include input-selection functions, retention or forgetting functions, output-modulation functions, or combinations thereof. The specific internal structure, number of layers, state dimensionality, and memory mechanisms employed are implementation-dependent and are not intended to limit the scope of the invention.

In some non-limiting examples, additional structural connections or auxiliary pathways may be used within the temporal modeling component to improve sensitivity to timing-dependent behaviors, such as detecting extended temporal patterns or slowly evolving neural oscillations. The use of such structures is optional and does not constrain the temporal modeling component to any particular internal configuration.

During operation of the temporal modeling component in the global processing path, sequential input representations may be processed iteratively across multiple time steps to capture longer-term temporal dependencies in the neural signals. In various embodiments, the temporal modeling component may maintain one or more internal state representations that are updated over time based on current input representations and prior state information, thereby enabling the modeling of temporal context and slowly evolving neural dynamics.

In certain non-limiting examples, the temporal modeling component may employ gating, state-update, or attention-based mechanisms configured to regulate the incorporation of new information, the retention or attenuation of prior information, and the generation of output representations at each time step. The specific mathematical formulations, activation functions, parameterizations, or internal structures of such mechanisms are implementation-dependent and are not intended to limit the scope of the invention.

After processing an input sequence over one or more time steps, the temporal modeling component may generate one or more output representations corresponding to the modeled long-term or global neural activity. Such output representations may include hidden state representations, aggregated state representations, embeddings, or other numerical descriptors that characterize sustained neural dynamics, contextual conditions, or inferred cognitive or physiological states.

In some embodiments, the output representations generated by the temporal modeling component may be further processed or mapped to higher-level descriptors representing estimated behavioral, cognitive, or physiological states of a subject. Such descriptors may include, for example, measures associated with attention, fatigue, engagement, or intent. The mapping may be performed using one or more post-processing operations, such as projection layers, normalization functions, probabilistic mappings, or other transformation mechanisms. The numerical values associated with such descriptors are illustrative only and are not intended to represent fixed or exclusive interpretations.

The one or more second feature representations described above may correspond to, be derived from, or incorporate such output representations of the temporal modeling component. The resulting second feature representations may be combined with one or more feature representations generated by other processing paths, such as the local processing path, to generate a final inference, prediction, or control signal.

Although, in certain examples illustrated in FIG. 2, the first and second processing paths may be implemented using a convolutional neural network and a long short-term memory network, respectively, other trained or untrained models may be employed in either processing path. The first trained model and the second trained model may each be selected from a variety of architectures capable of extracting short-term or long-term signal characteristics, including convolutional models, recurrent models, gated recurrent models, transformer-based models, temporal convolutional models, state-space models, or combinations thereof.

The specific architectures, layer configurations, state dimensionalities, numerical precisions, training frameworks, optimization techniques, or sequence lengths employed by the models in the first and second processing paths are implementation-dependent and are not intended to limit the scope of the invention. Accordingly, the dual-path processing framework described herein is not constrained to any particular model architecture, numerical configuration, or software implementation.

In some embodiments, a feedback-based error monitoring mechanism is employed to adaptively adjust parameters associated with the first processing path and the second processing path during operation. Each processing path performs feature extraction and generates one or more corresponding feature representations, including at least one first feature representation from the first processing path and at least one second feature representation from the second processing path.

Referring to FIG. 2, at block 218, the computing device may compare the at least one first feature representation and the at least one second feature representation at one or more times during operation to evaluate consistency, divergence, or complementary information between the processing paths. The comparison may be performed periodically, continuously, or in response to detected events, and may be based on one or more similarity, distance, error, confidence, or correlation measures.

Based on the comparison, the computing device generates a feedback signal indicative of a discrepancy, alignment, or confidence relationship between the processing paths. At block 220, the computing device adaptively adjusts one or more parameters associated with the first processing path, the second processing path, or both, based on the feedback signal.

Such parameter adjustments may include modifying processing weights, feature extraction parameters, temporal windowing characteristics, filtering parameters, or other configurable aspects of the processing paths.

In this manner, the first and second processing paths are adaptively coupled through a bidirectional feedback mechanism that enables the computing device to dynamically balance short-term and long-term neural signal representations, improve robustness to signal variability, and enhance overall inference or control performance. The specific comparison metrics, feedback generation logic, and adjustment strategies employed are implementation-dependent and are not intended to limit the scope of the invention.

In some embodiments, the comparison operation generates a feedback signal based on a relationship between the first feature representation and the second feature representation.

The feedback signal may be derived from one or more similarity measures, distance measures, error functions, confidence relationships, or correlation metrics that quantify alignment, divergence, or complementarity between the feature representations generated by the first and second processing paths.

In certain non-limiting examples, the feedback signal may be computed using a vector similarity measure, such as cosine similarity, between a first feature vector and a second feature vector. In other examples, the feedback signal may be computed using an error or discrepancy function, such as a mean squared error (MSE), a norm-based distance, or another loss function that reflects differences between the feature representations. The use of cosine similarity or mean squared error is illustrative only, and other similarity or error metrics may be employed.

Based on the feedback signal, the computing device adaptively adjusts one or more parameters associated with the first processing path, the second processing path, or both.

Such parameters may include, for example, signal selection parameters, feature fusion weights, temporal window characteristics, filtering parameters, or internal configuration parameters associated with feature extraction or temporal modeling components. The adjustments are performed to reduce discrepancies, improve alignment, or enhance complementary behavior between the processing paths in subsequent iterations.

In some embodiments, the adjustment process may include modifying both internal configuration parameters (such as weights or state-update characteristics) and external processing parameters (such as temporal window lengths or signal selection criteria). For example, the first processing path may adapt its sensitivity to short-term signal components in response to detected divergence from the second processing path, while the second processing path may adapt its temporal integration behavior to improve long-term contextual modeling when short-term representations are stable.

In certain implementations, the adaptive adjustment may be carried out within an iterative optimization or control loop. In one non-limiting example, parameter updates may be guided by gradient-based or rule-based optimization techniques, in which a parameter value is updated based on a feedback-derived adjustment signal. The magnitude and direction of such updates may be controlled by one or more adaptation coefficients, which may be fixed, learned, or dynamically adjusted based on performance metrics.

Threshold-based decision logic may be employed to determine when and how the first and second processing paths are adjusted. For example, when a similarity or discrepancy measure indicates insufficient alignment between short-term and long-term feature representations, the computing device may initiate adaptive parameter adjustments. For example, threshold-based decision logic may be employed to determine when and how the first and second processing paths are adjusted. For example, when a similarity or discrepancy measure indicates insufficient alignment between short-term and long-term feature representations, the computing device may initiate adaptive parameter adjustments. The specific threshold values, comparison criteria, and decision logic employed are implementation-dependent and are not intended to limit the scope of the invention. The specific threshold values, comparison criteria, and decision logic employed are implementation-dependent and are not intended to limit the scope of the invention.

In some embodiments, adaptive adjustments in the first processing path may include modifying signal selection or feature extraction characteristics to emphasize more reliable short-term neural signal components or suppress noise-related components. In other embodiments, adaptive adjustments in the second processing path may include modifying temporal integration or memory weighting behavior to improve responsiveness to changing neural dynamics. These adjustments enable the processing paths to dynamically rebalance sensitivity to short-term and long-term neural information.

For example, in the first processing path, when an alignment metric indicates poor correspondence between short-term and long-term representations, the computing device may modify one or more signal selection or feature extraction parameters in the first processing path to suppress noise-related or unreliable short-term neural signal components. Such adjustments may improve the quality of extracted short-term feature representations without requiring fixed frequency thresholds or model-specific constraints.

In some examples, the computing device may further adjust input conditioning, normalization, or weighting characteristics associated with the first processing path based on the feedback signal to reduce the influence of low-confidence or noise-dominated neural signal components. Such adjustments are illustrative and may be implemented in a variety of ways depending on system design.

In the second processing path, the computing device may adjust temporal integration behavior or contextual weighting characteristics based on the feedback signal to reduce the influence of outdated or less relevant long-term neural information and to increase responsiveness to recent neural dynamics. Such adjustments may be applied without directly manipulating internal model structures and are not limited to any particular temporal modeling architecture.

The threshold values, adaptation coefficients, and adjustment strategies described herein are provided for illustrative purposes only. In other embodiments, different values, ranges, or adaptive strategies may be employed, and such parameters may be learned, tuned, or dynamically adjusted during operation.

After one or more parameters in the first processing path and the second processing path have been adjusted, an adjusted first processing path and an adjusted second processing path are obtained. The computing device may then generate refined feature representations by reprocessing the neural signal components using the adjusted processing paths. The refined feature representations may be combined, fused, or otherwise utilized to generate an improved inference, prediction, or control signal.

In this manner, the computing device employs a bidirectional, feedback-driven coupling between the first and second processing paths, enabling continuous adaptation to neural signal variability, improved robustness across subjects and conditions, and enhanced performance of the overall system.

In some embodiments, the feedback mechanism may operate bidirectionally such that refined feature representations generated by one processing path are provided as contextual input to the other processing path to influence subsequent processing behavior. In this manner, adaptations applied in one processing path may indirectly influence the behavior of the other processing path in subsequent iterations without requiring direct manipulation of internal model structures.

In some examples, adjustments applied to the second processing path may involve modifying temporal integration behavior, state-update weighting, or contextual emphasis based on refined feature representations received from the first processing path. Similarly, adjustments applied to the first processing path may involve modifying feature extraction sensitivity or input conditioning behavior based on refined feature representations received from the second processing path. Through this bidirectional feedback and parameter adaptation, the computing device achieves improved consistency and accuracy between the processing paths.

In one practical embodiment, the computing device may be applied to control of a prosthetic limb or other assistive device. Neural signals obtained from a subject may be processed using the dual-path framework described herein, in which neural signal components associated with relatively rapid variations are processed in a first processing path and neural signal components associated with more slowly evolving dynamics are processed in a second processing path.

After feature extraction, the computing device may evaluate a relationship between feature representations generated by the first and second processing paths. In one non-limiting example, a similarity or discrepancy measure derived from the feature representations may indicate insufficient alignment between short-term and long-term neural information, suggesting that the two processing paths are not optimally synchronized.

In response to such a condition, the computing device may adaptively adjust parameters associated with both processing paths. For example, the first processing path may adjust signal selection or filtering characteristics to suppress noise-related or unreliable short-term neural signal components, while the second processing path may adjust temporal integration or contextual weighting characteristics to reduce the influence of outdated or irrelevant long-term trends and increase responsiveness to recent neural activity.

After the parameter adjustments, refined feature representations generated by the first processing path may be provided to the second processing path or otherwise incorporated into subsequent processing cycles, enabling the processing paths to co-adapt based on updated neural signal characteristics. Through this feedback-driven co-adaptation, the computing device can achieve more stable, synchronized, and accurate decoding of neural signals across multiple temporal abstractions, thereby improving control performance of the prosthetic limb or other assistive device.

The specific similarity measures, adjustment magnitudes, parameter types, and adaptation strategies described herein are provided for illustrative purposes only, and other values or strategies may be employed without departing from the scope of the invention.

In some embodiments, as shown at block 222 of FIG. 2, the method 200 may further include a fusion process configured to combine the at least one refined first feature representation and the at least one refined second feature representation to generate a fused feature representation or feature matrix. The fused feature representation may be used to determine a neural activity pattern representing a cognitive, physiological, or motor intent state of a user.

In some embodiments, fusing the at least one refined first feature representation and the at least one refined second feature representation may comprise determining respective weighting factors associated with the refined first feature representation and the refined second feature representation. The weighting factors may be determined based on one or more confidence metrics, reliability indicators, or quality measures associated with the processing paths that generated the respective feature representations.

In various non-limiting examples, the first processing path and the second processing path may each generate a confidence value associated with their respective refined feature representations. Such confidence values may be derived from model-specific quality scores, historical performance indicators, prediction variance, stability measures, or other metrics reflecting the reliability or consistency of each processing path. The confidence values may be normalized, scaled, or otherwise transformed prior to use in the fusion process.

The fusion process may generate a combined or fused feature representation by applying a weighted combination, aggregation, or integration of the refined first feature representation and the refined second feature representation according to the determined weighting factors. The resulting fused feature representation may represent a consolidated characterization of neural signal information across multiple temporal abstractions.

In some embodiments, the fusion process may generate a multi-dimensional feature structure that includes contributions from both the refined first feature representation and the refined second feature representation. Such a feature structure may include dimensions corresponding to channels, feature types, temporal context, confidence measures, or other attributes relevant to downstream inference or control. The specific dimensionality, structure, and representation of the fused feature data are implementation-dependent and are not intended to limit the scope of the invention.

In certain implementations, the computing device may maintain a rolling or sliding temporal buffer of fused feature representations, enabling continuous updating of the fused representation as new neural signal data is processed. This allows the system to maintain temporal continuity and responsiveness during ongoing operation.

In some embodiments, the computing device may optionally include a task-adaptive weighting or fusion module configured to further adjust the relative contributions of the refined first feature representation and the refined second feature representation based on application context, task requirements, or operating conditions. Such a fusion module may be implemented using a rule-based mechanism, a trained model, or a combination thereof, and may dynamically adjust weighting behavior to emphasize short-term or long-term neural information as appropriate for a given application.

The specific weighting strategies, confidence normalization techniques, fusion mechanisms, and adaptation parameters described herein are provided for illustrative purposes only. Other fusion approaches, confidence measures, or weighting schemes may be employed without departing from the scope of the invention.

After fusion, the computing device may transmit the resulting fused feature representation (e.g., a fused feature matrix or fused feature vector) to external devices or subsystems. Such external devices may include, for example, a robotic or prosthetic limb, a visualization interface, or a neurostimulation system. The fused representation provides an integrated characterization of both immediate neural activity and longer-term state information, enabling improved decoding of user intent and more stable real-time control.

According to another aspect of this disclosure, a computing device for processing neural signals associated with biological brain activities is provided. FIG. 3 is a block diagram of a computing device 300 for processing neural signals associated with biological brain activities in accordance with examples disclosed herein. As shown in FIG. 3, the computing device 300 includes an electrode array 310 and a semiconductor device 320 comprising at least one processor and memory. The electrode array 310 is configured to obtain a plurality of neural signals. The semiconductor device 320 is configured to separate the plurality of neural signals into components associated with different temporal characteristics, process the components associated with relatively short-term dynamics in a first processing path 322 to generate at least one first feature, and process the components associated with relatively long-term dynamics in a second processing path 323 to generate at least one second feature. A neural activity pattern is determined based on the at least one first feature and the at least one second feature.

The semiconductor device 320 may be implemented as a chip including electrical circuits. The chip may be fabricated using complementary metal-oxide semiconductor (CMOS) technology (or other semiconductor technologies) and may integrate multiple functional modules configured to perform neural signal acquisition and on-chip data processing. As shown in FIG. 3, the chip may incorporate a preprocess circuit 321. The preprocess circuit 321 may include analog-to-digital converters (ADCs) 331 and low-noise front-end amplifier circuits 332 configured to condition raw neural signals from the electrode array 310, suppress noise and interference, and preserve signal integrity. In some examples, the integrated front-end amplifiers 332 may include programmable gain stages, filters, and impedance-matching circuitry to accommodate varying signal amplitudes and sensing electrode characteristics. The ADCs may be configured to support multi-channel conversion to enable simultaneous acquisition of neural signals.

In some embodiments, the at least one first feature comprises at least one first vector. As shown in FIG. 3, the semiconductor device 320 may further comprise a first processor 341 disposed in the first processing path 322. The first processor 341 is configured to input neural signal components associated with relatively short-term dynamics (also referred to as first components) into a first trained model deployed on the first processor 341 in the first processing path 322, and to generate the at least one first vector based on the first trained model. In some non-limiting examples, the first trained model 352 may comprise a convolutional neural network (e.g., a CNN model) or another feature extraction model.

In some embodiments, the at least one second feature comprises at least one second vector. As shown in FIG. 3, the semiconductor device 320 may further comprise a second processor 342 disposed in the second processing path 323. The second processor 342 is configured to input neural signal components associated with relatively long-term dynamics into a second trained model deployed on the second processor 342 in the second processing path 323, and to generate the at least one second vector based on the second trained model. In some examples, the second processing path may include a resampling, aggregation, or temporal conditioning unit (e.g., a downsampling unit 361 shown in FIG. 3) to generate suitable representations for the second trained model. In some non-limiting examples, the second trained model 362 may comprise a long short-term memory network or another temporal modeling component capable of capturing extended temporal dependencies (e.g., an LSTM model). The first and second processors 341 and 342 may be dedicated accelerators or general-purpose processing units.

In some embodiments, the semiconductor device 320 separates neural signal components associated with different temporal characteristics using spectral decomposition, time-frequency analysis, filtering, or learned representation techniques, including but not limited to FFT, wavelet transforms, adaptive filter banks, or learned spectral filters. As shown in FIG. 3, the first processing path 322 may include a signal decomposition circuit configured to generate neural signal representations suitable for separating components associated with different temporal characteristics, wherein an FFT circuit 351 may be used in some embodiments as one example of a spectral decomposition block. The use of FFT is illustrative and does not limit the separation mechanism.

In some embodiments, the semiconductor device 320 is further configured to compare the at least one first feature and the at least one second feature to generate a feedback signal, and to adjust at least one parameter associated with the first processing path 322 and the second processing path 323 based on the feedback signal to obtain an adjusted first processing path 322 and an adjusted second processing path 323, respectively. Still referring to FIG. 3, the feedback and output circuit 324 may compare the first feature and the second feature and generate a feedback signal based on the comparison result. The feedback signal may be provided to the first processing path 322 and the second processing path 323 to adjust one or more parameters associated with the two paths.

In some embodiments, the semiconductor device 320 compares the at least one first feature and the at least one second feature by determining at least one similarity metric, discrepancy metric, confidence metric, or error metric between the at least one first vector and the at least one second vector, including cosine similarity and/or mean squared error in some non-limiting examples.

In some embodiments, the at least one parameter comprises one or more signal selection parameters, feature fusion parameters, temporal processing parameters, filtering parameters, or internal configuration parameters associated with the first processing path or the second processing path.

In some embodiments, the semiconductor device 320 adjusts at least one parameter in the first processing path 322 by modifying one or more signal selection parameters or feature extraction characteristics in response to the feedback signal indicating insufficient alignment between the first and second processing paths.

In some embodiments, the semiconductor device 320 adjusts at least one parameter in the second processing path 323 by modifying one or more temporal integration or contextual weighting characteristics in response to the feedback signal indicating insufficient alignment between the first and second processing paths.

In some embodiments, the semiconductor device 320 is further configured to reconstruct at least one refined first feature and at least one refined second feature based on the adjusted first processing path 322 and adjusted second processing path 323, respectively.

In some embodiments, the semiconductor device 320 adjusts at least one parameter in the first processing path 322 and the second processing path 323 by providing at least one refined first feature representation as contextual input to the second processing path 323 and by providing at least one refined second feature representation as contextual input to the first processing path 322, thereby enabling bidirectional adaptation between the processing paths.

In some embodiments, the semiconductor device 320 is further configured to fuse the at least one refined first feature and the at least one refined second feature into a feature matrix or other fused feature representation, wherein the neural activity pattern is determined based on the fused feature representation.

In some embodiments, the semiconductor device 320 fuses the at least one refined first feature and the at least one refined second feature by determining respective weighting factors based on one or more confidence metrics, and combining the refined feature representations according to the weighting factors.

In some embodiments, the computing device 300 further comprises a filter configured to preprocess the plurality of neural signals to filter out noise components.

In some embodiments, the neural activity pattern is determined to control a prosthetic device.

In some embodiments, the electrode array 310 comprises a plurality of channels.

In some embodiments, the computing device 300 is configured for low-latency and energy-efficient operation.

In some embodiments, the computing device 300 is a wearable or an implantable device.

In certain embodiments, the computing device 300 may be conceptualized as two temporal chains arranged in a helical or interleaving topology, where each processing path (e.g., the first processing path 322 and the second processing path 323) corresponds to one chain (e.g., Chain A and Chain B). Cross-points between the chains represent synchronization landmarks used for comparing features, generating feedback signals, or updating model parameters. This conceptual representation may be applied to scheduling, feature alignment, phase normalization, or fusion operations. The helical mapping is optional and does not constrain the computing device 300 to any physical helical structure.

It should be noted that the described techniques include possible implementations, and that the operations and the blocks may be rearranged, reordered, or otherwise modified and that other implementations are possible. Further, portions from two or more of the methods may be combined.

Information and signals described herein may be represented using any of a variety of different technologies and techniques. For example, data, instructions, commands, information, signals, bits, or symbols of signaling that may be referenced throughout the above description may be represented by voltages, currents, electromagnetic waves, magnetic fields or particles, optical fields or particles, or any combination thereof. Some drawings may illustrate signals as a single signal; however, the signal may represent a bus of signals, where the bus may have a variety of bit widths.

The terms “electronic communication,” “conductive contact,” “connected,” and “coupled” may refer to a relationship between components that supports the flow of signals between the components. Components are considered in electronic communication with (or in conductive contact with or connected with or coupled with) one another if there is any conductive path between the components that can, at any time, support the flow of signals between the components. At any given time, the conductive path between components that are in electronic communication with each other (or in conductive contact with or connected with or coupled with) may be an open circuit or a closed circuit based on the operation of the device that includes the connected components. The conductive path between connected components may be a direct conductive path between the components or the conductive path between connected components may be an indirect conductive path that may include intermediate components, such as switches, transistors, or other components. In some examples, the flow of signals between the connected components may be interrupted for a time, for example, using one or more intermediate components such as switches or transistors.

The term “coupling” (e.g., “electrically coupling”) may refer to a condition of moving from an open-circuit relationship between components in which signals are not presently capable of being communicated between the components over a conductive path to a closed-circuit relationship between components in which signals are capable of being communicated between components over the conductive path. If a component, such as a controller, couples other components together, the component initiates a change that allows signals to flow between the other components over a conductive path that previously did not permit signals to flow.

Processors described herein may be implemented as CPUs, GPUs, DSPs, ASICs, FPGAs, systolic arrays, or hybrid accelerators. References to specific hardware blocks (e.g., FFT unit, CNN engine, LSTM engine) are provided for illustration and do not limit the architecture to any particular implementation.

The term “isolated” refers to a relationship between components in which signals are not presently capable of flowing between the components. Components are isolated from each other if there is an open circuit between them. For example, two components separated by a switch that is positioned between the components are isolated from each other if the switch is open. If a controller isolates two components, the controller affects a change that prevents signals from flowing between the components using a conductive path that previously permitted signals to flow.

The terms “if,” “when,” “based on,” or “based at least in part on” may be used interchangeably. In some examples, if the terms “if,” “when,” “based on,” or “based at least in part on” are used to describe a conditional action, a conditional process, or connection between portions of a process, the terms may be interchangeable.

The term “in response to” may refer to one condition or action occurring at least partially, if not fully, as a result of a previous condition or action. For example, a first condition or action may be performed and second condition or action may at least partially occur as a result of the previous condition or action occurring (whether directly after or after one or more other intermediate conditions or actions occurring after the first condition or action).

The devices discussed herein may be formed on a semiconductor substrate, such as silicon, germanium, silicon-germanium alloy, gallium arsenide, gallium nitride, etc. In some examples, the substrate is a semiconductor wafer. In some other examples, the substrate may be a silicon-on-insulator (SOI) substrate, such as silicon-on-glass (SOG) or silicon-on-sapphire (SOP), or epitaxial layers of semiconductor materials on another substrate. The conductivity of the substrate, or sub-regions of the substrate, may be controlled through doping using various chemical species including, but not limited to, phosphorous, boron, or arsenic. Doping may be performed during the initial formation or growth of the substrate, by ion-implantation, or by any other doping means.

The description set forth herein, in connection with the appended drawings, describes example configurations and does not represent all the examples that may be implemented or that are within the scope of the claims. The term “exemplary” used herein means “serving as an example, instance, or illustration” and not “preferred” or “advantageous over other examples.” The detailed description includes specific details to provide an understanding of the described techniques. These techniques, however, may be practiced without these specific details. In some instances, well-known structures and devices are shown in block diagram form to avoid obscuring the concepts of the described examples.

In the appended figures, similar components or features may have the same reference label. Further, various components of the same type may be distinguished by following the reference label by a hyphen and a second label that distinguishes among the similar components. If just the first reference label is used in the specification, the description is applicable to any one of the similar components having the same first reference label irrespective of the second reference label.

As used herein, including in the claims, “or” as used in a list of items (for example, a list of items prefaced by a phrase such as “at least one of” or “one or more of”) indicates an inclusive list such that, for example, a list of at least one of A, B, or C means A or B or C or AB or AC or BC or ABC (i.e., A and B and C). Also, as used herein, the phrase “based on” shall not be construed as a reference to a closed set of conditions. For example, an exemplary step that is described as “based on condition A” may be based on both a condition A and a condition B without departing from the scope of the present disclosure. In other words, as used herein, the phrase “based on” shall be construed in the same manner as the phrase “based at least in part on.”

The description herein is provided to enable a person skilled in the art to make or use the disclosure. Various modifications to the disclosure will be apparent to those skilled in the art, and the generic principles defined herein may be applied to other variations without departing from the scope of the disclosure. Thus, the disclosure is not limited to the examples and designs described herein but is to be accorded the broadest scope consistent with the principles and novel features disclosed herein.

Example 1. A method performed by a computing device for processing neural signals associated with biological brain activities, comprising:
obtaining a plurality of neural signals, the plurality of neural signals comprising first neural signal components associated with relatively short-term variations and second neural signal components associated with relatively long-term variations;
separating the first neural signal components from the second neural signal components;
processing the first neural signal components in a first processing path to generate at least one first feature indicative of short-term neural activity; and
processing the second neural signal components in a second processing path to generate at least one second feature indicative of long-term neural activity;
wherein a neural activity pattern is determined based on the at least one first feature and the at least one second feature.

Example 2. The method of example 1, wherein the at least one first feature comprises at least one first vector, and wherein processing the first neural signal components comprises:
inputting the first neural signal components into a first trained model in the first processing path; and
generating the at least one first vector based on the first trained model.

Example 3. The method of example 1 or 2, wherein the at least one second feature comprises at least one second vector, and wherein processing the second neural signal components comprises:
inputting the second neural signal components into a second trained model in the second processing path; and
generating the at least one second vector based on the second trained model.

Example 4. The method of any of examples 1-3, wherein separating the first neural signal components from the second neural signal components comprises:
applying a spectral, temporal, or time-frequency decomposition.

Example 5. The method of any of examples 1-4, further comprising:
comparing the at least one first feature and the at least one second feature to generate a feedback signal; and
adjusting at least one parameter in the first processing path and the second processing path based on the feedback signal to obtain an adjusted first processing path and an adjusted second processing path, respectively.

Example 6. The method of any of examples 1-5, wherein comparing the at least one first feature and the at least one second feature to generate the feedback signal comprises:
determining at least one similarity metric, discrepancy metric, confidence metric, or error metric between the at least one first vector and the at least one second vector.

Example 7. The method of any of examples 1-6, wherein the at least one parameter comprises one or more signal selection parameters, feature fusion parameters, temporal processing parameters, filtering parameters, or internal model parameters associated with the first processing path or the second processing path.

Example 8. The method of any of examples 1-7, wherein adjusting at least one parameter in the first processing path comprises:
modifying one or more signal selection parameters or feature extraction parameters in response to the feedback signal indicating insufficient alignment between the first processing path and the second processing path.

Example 9. The method of any of examples 1-8, wherein adjusting at least one parameter in the second processing path comprises:
modifying one or more temporal processing parameters or temporal modeling parameters in response to the feedback signal indicating insufficient alignment between the first processing path and the second processing path.

Example 10. The method of any of examples 1-9, further comprising:
reconstructing at least one refined first feature and at least one refined second feature based on the adjusted first processing path and the adjusted second processing path, respectively.

Example 11. The method of any of examples 1-10, wherein adjusting at least one parameter in the first processing path and the second processing path further comprises:
providing at least one refined first feature representation as contextual input to the second processing path; and
providing at least one refined second feature representation as contextual input to the first processing path.

Example 12. The method of any of examples 1-11, further comprising:
fusing the at least one refined first feature and the at least one refined second feature into a fused feature representation, wherein the neural activity pattern is determined based on the fused feature representation.

Example 13. The method of any of examples 1-12, wherein fusing the at least one refined first feature and the at least one refined second feature comprises:
determining a first weight for the at least one refined first feature based on a confidence metric;
determining a second weight for the at least one refined second feature based on the confidence metric; and
fusing the at least one refined first feature and the at least one refined second feature based on the first weight and the second weight.

Example 14. The method of any of examples 1-13, further comprising:
preprocessing the plurality of neural signals to filter out noise components.

Example 15. The method of any of examples 1-14, wherein the neural activity pattern is determined to control a prosthetic device.

Example 16. The method of any of examples 1-15, wherein the first trained model and the second trained model are each selected from the group consisting of:
a convolutional neural network (CNN);
a long short-term memory (LSTM) network;
a gated recurrent unit (GRU) network;
a transformer block; or
a temporal convolutional network.

Example 17. A computing device for processing neural signals associated with biological brain activities comprising:
an electrode array configured to obtain a plurality of neural signals, the plurality of neural signals comprising first neural signal components associated with relatively short-term variations and second neural signal components associated with relatively long-term variations; and
a semiconductor device comprising at least one processor and memory, the semiconductor device being configured to:
   separate the first neural signal components from the second neural signal components;
   process the first neural signal components in a first processing path to generate at least one first feature indicative of short-term neural activity; and
   process the second neural signal components in a second processing path to generate at least one second feature indicative of long-term neural activity;
   wherein a neural activity pattern is determined based on the at least one first feature and the at least one second feature.

Example 18. The computing device of example 17, wherein the at least one first feature comprises at least one first vector, and the semiconductor device further comprises:
a first processor disposed in the first processing path and configured to:
   input the first neural signal components into a first trained model deployed on the first processor in the first processing path; and
   generate the at least one first vector based on the first trained model.

Example 19. The computing device of example 17 or 18, wherein the at least one second feature comprises at least one second vector, and the semiconductor device further comprises:
a second processor disposed in the second processing path and configured to:
   input the second neural signal components into a second trained model deployed on the second processor in the second processing path; and
   generate the at least one second vector based on the second trained model.

Example 20. The computing device of any of examples 17-19, wherein the semiconductor device separates the first neural signal components from the second neural signal components by applying a spectral, temporal, or time-frequency decomposition.

Example 21. The computing device of any of examples 17-20, wherein the semiconductor device is further configured to:
compare the at least one first feature and the at least one second feature to generate a feedback signal; and
adjust at least one parameter in the first processing path and the second processing path based on the feedback signal to obtain an adjusted first processing path and an adjusted second processing path, respectively.

Example 22. The computing device of any of examples 17-21, wherein the semiconductor device compares the at least one first feature and the at least one second feature by determining at least one similarity metric, discrepancy metric, confidence metric, or error metric between the at least one first vector and the at least one second vector.

Example 23. The computing device of any of examples 17-22, wherein the at least one parameter comprises one or more signal selection parameters, feature fusion parameters, temporal processing parameters, filtering parameters, or internal model parameters associated with the first processing path or the second processing path.

Example 24. The computing device of any of examples 17-23, wherein the semiconductor device adjusts at least one parameter in the first processing path by modifying one or more signal selection parameters or feature extraction parameters in response to the feedback signal indicating insufficient alignment between the first processing path and the second processing path.

Example 25. The computing device of any of examples 17-24, wherein the semiconductor device adjusts at least one parameter in the second processing path by modifying one or more temporal processing parameters or temporal modeling parameters in response to the feedback signal indicating insufficient alignment between the first processing path and the second processing path.

Example 26. The computing device of any of examples 17-25, wherein the semiconductor device is further configured to reconstruct at least one refined first feature and at least one refined second feature based on the adjusted first processing path and the adjusted second processing path, respectively.

Example 27. The computing device of any of examples 17-26, wherein the semiconductor device adjusts at least one parameter in the first processing path and the second processing path by:
providing at least one refined first feature representation as contextual input to the second processing path; and
providing at least one refined second feature representation as contextual input to the first processing path.

Example 28. The computing device of any of examples 17-27, wherein the semiconductor device is further configured to fuse the at least one refined first feature and the at least one refined second feature into a fused feature representation, wherein the neural activity pattern is determined based on the fused feature representation.

Example 29. The computing device of any of examples 17-28, wherein the semiconductor device fuses the at least one refined first feature and the at least one refined second feature by:
determining a first weight for the at least one refined first feature based on a confidence metric;
determining a second weight for the at least one refined second feature based on the confidence metric; and
fusing the at least one refined first feature and the at least one refined second feature based on the first weight and the second weight.

Example 30. The computing device of any of examples 17-29, further comprising:
a filter configured to preprocess the plurality of neural signals to filter out noise components.

Example 31. The computing device of any of examples 17-30, wherein the neural activity pattern is determined to control a prosthetic device.

Example 32. The computing device of any of examples 17-31, wherein the electrode array comprises a plurality of channels ..

Example 33. The computing device of any of examples 17-32, wherein the computing device is configured for low-latency and energy-efficient operation.

Example 34. The computing device of any of examples 17-33, wherein the computing device is a wearable or an implantable device.

Example 35. The computing device of any of examples 17-34, wherein the first trained model and the second trained model are each selected from the group consisting of:
a convolutional neural network (CNN);
a long short-term memory (LSTM) network;
a gated recurrent unit (GRU) network;
a transformer block; or
a temporal convolutional network.

1. A method performed by a computing device for processing neural signals associated with biological brain activities, comprising:
obtaining a plurality of neural signals, the plurality of neural signals comprising first neural signal components associated with relatively short-term variations and second neural signal components associated with relatively long-term variations;
separating the first neural signal components from the second neural signal components;
processing the first neural signal components in a first processing path to generate at least one first feature indicative of short-term neural activity; and
processing the second neural signal components in a second processing path to generate at least one second feature indicative of long-term neural activity;
wherein a neural activity pattern is determined based on the at least one first feature and the at least one second feature.

2. The method of claim 1, wherein the at least one first feature comprises at least one first vector, and wherein processing the first neural signal components comprises:
inputting the first neural signal components into a first trained model in the first processing path; and
generating the at least one first vector based on the first trained model.

3. The method of claim 2, wherein the at least one second feature comprises at least one second vector, and wherein processing the second neural signal components comprises:
inputting the second neural signal components into a second trained model in the second processing path; and
generating the at least one second vector based on the second trained model.

4. The method of claim 1, wherein separating the first neural signal components from the second neural signal components comprises:
applying a spectral, temporal, or time-frequency decomposition.

5. The method of claim 3, further comprising:
comparing the at least one first feature and the at least one second feature to generate a feedback signal; and
adjusting at least one parameter in the first processing path and the second processing path based on the feedback signal to obtain an adjusted first processing path and an adjusted second processing path, respectively.

6. The method of claim 5, wherein comparing the at least one first feature and the at least one second feature to generate the feedback signal comprises:
determining at least one similarity metric, discrepancy metric, confidence metric, or error metric between the at least one first vector and the at least one second vector.

7. The method of claim 6, wherein the at least one parameter comprises one or more signal selection parameters, feature fusion parameters, temporal processing parameters, filtering parameters, or internal model parameters associated with the first processing path or the second processing path.

8. The method of claim 7, wherein adjusting at least one parameter in the first processing path comprises:
modifying one or more signal selection parameters or feature extraction parameters in response to the feedback signal indicating insufficient alignment between the first processing path and the second processing path.

9. The method of claim 7, wherein adjusting at least one parameter in the second processing path comprises:
modifying one or more temporal processing parameters or temporal modeling parameters in response to the feedback signal indicating insufficient alignment between the first processing path and the second processing path.

10. The method of claim 5, further comprising:
reconstructing at least one refined first feature and at least one refined second feature based on the adjusted first processing path and the adjusted second processing path, respectively.

11. The method of claim 10, wherein adjusting at least one parameter in the first processing path and the second processing path further comprises:
providing at least one refined first feature representation as contextual input to the second processing path; and
providing at least one refined second feature representation as contextual input to the first processing path.

12. The method of claim 10, further comprising:
fusing the at least one refined first feature and the at least one refined second feature into a fused feature representation, wherein the neural activity pattern is determined based on the fused feature representation.

13. The method of claim 12, wherein fusing the at least one refined first feature and the at least one refined second feature comprises:
determining a first weight for the at least one refined first feature based on a confidence metric;
determining a second weight for the at least one refined second feature based on the confidence metric; and
fusing the at least one refined first feature and the at least one refined second feature based on the first weight and the second weight.

14. The method of claim 1, further comprising:
preprocessing the plurality of neural signals to filter out noise components.

15. The method of claim 1, wherein the neural activity pattern is determined to control a prosthetic device.

16. The method of claim 3, wherein the first trained model and the second trained model are each selected from the group consisting of:
a convolutional neural network (CNN);
a long short-term memory (LSTM) network;
a gated recurrent unit (GRU) network;
a transformer block; or
a temporal convolutional network.

17. A computing device for processing neural signals associated with biological brain activities comprising:
an electrode array configured to obtain a plurality of neural signals, the plurality of neural signals comprising first neural signal components associated with relatively short-term variations and second neural signal components associated with relatively long-term variations; and
a semiconductor device comprising at least one processor and memory, the semiconductor device being configured to:
separate the first neural signal components from the second neural signal components;
process the first neural signal components in a first processing path to generate at least one first feature indicative of short-term neural activity; and
process the second neural signal components in a second processing path to generate at least one second feature indicative of long-term neural activity;
wherein a neural activity pattern is determined based on the at least one first feature and the at least one second feature.

18. The computing device of claim 17, wherein the at least one first feature comprises at least one first vector, and the semiconductor device further comprises:
a first processor disposed in the first processing path and configured to:
input the first neural signal components into a first trained model deployed on the first processor in the first processing path; and
generate the at least one first vector based on the first trained model.

19. The computing device of claim 18, wherein the at least one second feature comprises at least one second vector, and the semiconductor device further comprises:
a second processor disposed in the second processing path and configured to:
input the second neural signal components into a second trained model deployed on the second processor in the second processing path; and
generate the at least one second vector based on the second trained model.

20. The computing device of claim 17, wherein the semiconductor device separates the first neural signal components from the second neural signal components by applying a spectral, temporal, or time-frequency decomposition.

21. The computing device of claim 19, wherein the semiconductor device is further configured to:
compare the at least one first feature and the at least one second feature to generate a feedback signal; and
adjust at least one parameter in the first processing path and the second processing path based on the feedback signal to obtain an adjusted first processing path and an adjusted second processing path, respectively.

22. The computing device of claim 21, wherein the semiconductor device compares the at least one first feature and the at least one second feature by determining at least one similarity metric, discrepancy metric, confidence metric, or error metric between the at least one first vector and the at least one second vector.

23. The computing device of claim 22, wherein the at least one parameter comprises one or more signal selection parameters, feature fusion parameters, temporal processing parameters, filtering parameters, or internal model parameters associated with the first processing path or the second processing path.

24. The computing device of claim 23, wherein the semiconductor device adjusts at least one parameter in the first processing path by modifying one or more signal selection parameters or feature extraction parameters in response to the feedback signal indicating insufficient alignment between the first processing path and the second processing path.

25. The computing device of claim 23, wherein the semiconductor device adjusts at least one parameter in the second processing path by modifying one or more temporal processing parameters or temporal modeling parameters in response to the feedback signal indicating insufficient alignment between the first processing path and the second processing path.

26. The computing device of claim 21, wherein the semiconductor device is further configured to reconstruct at least one refined first feature and at least one refined second feature based on the adjusted first processing path and the adjusted second processing path, respectively.

27. The computing device of claim 26, wherein the semiconductor device adjusts at least one parameter in the first processing path and the second processing path by:
providing at least one refined first feature representation as contextual input to the second processing path; and
providing at least one refined second feature representation as contextual input to the first processing path.

28. The computing device of claim 26, wherein the semiconductor device is further configured to fuse the at least one refined first feature and the at least one refined second feature into a fused feature representation, wherein the neural activity pattern is determined based on the fused feature representation.

29. The computing device of claim 28, wherein the semiconductor device fuses the at least one refined first feature and the at least one refined second feature by:
determining a first weight for the at least one refined first feature based on a confidence metric;
determining a second weight for the at least one refined second feature based on the confidence metric; and
fusing the at least one refined first feature and the at least one refined second feature based on the first weight and the second weight.

30. The computing device of claim 17, further comprising:
a filter configured to preprocess the plurality of neural signals to filter out noise components.

31. (canceled)

32. (canceled)

33. (canceled)

34. (canceled)

35. (canceled)
